(19) United States
(12) Patent Application Publication
(10) Pub. No.: US 2023/0405069 A1
(43) Pub. Date: Dec. 21, 2023

(54) METHODS OF TREATING AND PREVENTING INFECTIONS OF CORONAVIRUS AND OTHER INDICATIONS, AND COMPOSITIONS FOR USE WITH SAME

(71) Applicants: Pentibo Nagaipe Baihua MIIPO, New York, NY (US); Martin Orengo Baihua MIIPO, New York, NY (US)

(72) Inventors: Pentibo Nagaipe Baihua MIIPO, New York, NY (US); Martin Orengo Baihua MIIPO, New York, NY (US)

(21) Appl. No.: 18/037,716

(22) PCT Filed: Nov. 19, 2021
(86) PCT No.: PCT/IB2021/060772

§ 371 Date: May 18, 2023

Related U.S. Application Data
(60) Provisional application No. 63/116,531, filed on Nov. 20, 2020.

(51) Int. Cl. A61K 36/48; A61K 36/53; A61K 36/74; A61K 36/24; A61P 31/04

(52) U.S. Cl. CPC A61K 36/48; A61K 36/53; A61K 36/74; A61K 36/24; A61P 31/04; A61K 2236/331

(57) ABSTRACT

Disclosed herein are compositions and methods for treating one or more symptoms of viral infection or preventing same. The disclosed compositions may include natural or synthetic compounds that are the similar or the same as compounds found in one or more plants or trees of Ecuador. In many embodiments, the one or more plants or trees are selected from Balsamo tree, Canaleta tree, Ajo de Monte, and Uña de Gato.

### CROSS-REFERENCE TO RELATED APPLICATIONS

This application claims benefit of priority pursuant to 35 U.S.C. § 119(e) of U.S. provisional patent application No. 63/116,531 entitled “Methods of Treating and Preventing Infections of Coronavirus and Other Indications, and Compositions For Use With Same,” filed on Nov. 20, 2020, which is hereby incorporated by reference in its entirety.

### FIELD

The disclosed processes, methods, and compositions are directed to treating and preventing viral infections and symptoms thereof in a patient, and/or eliminating virus from the patient, for example subjects with infections from coronavirus, influenza, and other viral pathogens, or at risk thereof.

### BACKGROUND

There are many mammalian viruses, some of which are pathogenic in humans. Viruses may be DNA- or RNA-based, and also vary in size, infection rate, mortality, mutation rate, etc. Pathogenic human viruses include the causative agents of seasonal flu, polio, measles, Ebola, smallpox, chickenpox, rubella, etc.

One type of pathogenic virus is the causative agent of a recent pandemic that threatens to eclipse the Spanish Flu Pandemic of 1918 in terms of mortality rate and total mortality. That virus belongs to the coronavirus (CoV) family, a group of mammalian and avian enveloped RNA viruses. Coronaviruses infect the host's respiratory tract resulting in asymptomatic presentation to mild symptoms to hospitalization to death. It also appears to target kidney cells, liver cells, blood vessels, and even the brain, leading to myriad symptoms including neurological symptoms. Coronavirus infections often present as a bout of common cold. Lethal varieties of the CoV family are the causative agent in Middle East Respiratory Syndrome (MERS-CoV), Severe Acute Respiratory Syndrome (SARS-CoV) and the 2019 pandemic, COVID-19.

Coronaviruses may be transmitted between animals and humans. Common signs of infection include respiratory symptoms, fever, chills, headache, cough, shortness of breath, weakness, dry cough, vomiting, weight loss, diarrhea, and breathing difficulties. In more severe cases, infection can cause pneumonia, severe acute respiratory syndrome, kidney/organ failure, and even death.

There is a need for patient treatments directed to preventing coronavirus infections and/or lessening their effects and/or eliminating virus from a previously infected patient.

### SUMMARY

Disclosed herein are compositions and methods for preventing, treating, and/or curing viral infections comprising the use of one or more compounds derived from a botanical source selected from one or more of a Balsamo tree (Myroxylon balsamum), Canaleta tree or Canalete Tree (Aspidosperma rigidum), Ajo de Monte plant (Mansoa alliacea), and Uña de Gato (Uncaria guianensis). In many embodiments the composition may include a first compound in a first solvent, wherein the first compound is obtained from a part of the Balsamo tree. In other embodiments, the first compound is obtained from a part of the Canaleta tree. The disclosed compositions may also include a second compound in a second solvent, a third compound in a third solvent, and/or a fourth compound in a fourth solvent, wherein the second, third, and/or fourth compounds are obtained from a botanical source. Where the first compound is from a part of the Balsamo tree the botanical sources of the second, third, or fourth compounds may be selected from Canaleta tree, Ajo de Monte, and Uña de Gato, and where the first compound is from a part of the Canaleta tree, the botanical source of the second, third, and/or fourth compounds may be selected from Balsamo tree, Ajo de Monte, and Uña de Gato. In many embodiments, the part of the Balsamo tree or the Canaleta tree is selected from tree sap, tree bark, roots, and/or any other plant part. The viral infection may be caused by various agents including coronavirus, SARS Coronavirus-19, influenza, etc. In many embodiments, the amount of the first, second, third, or fourth solvent may be between about 3 and about 150 ml, and the solvents may be the same or separate solvents for each compound. In many embodiments, the composition may be administered to a subject and may result in the subject being free symptoms of a viral infection and/or of the virus itself, as demonstrated by at least one method of identifying a viral infection.

Also disclosed herein are compositions for treating viral infection, preventing a viral infection, or curing a viral infection, the composition comprising a first compound in a first solvent; and a second compound in a second solvent, wherein the first and/or second compounds are obtained from a botanical source. The disclosed compositions may include one or more of a third compound in a third solvent and a fourth compound in a fourth solvent, wherein the third and/or fourth compounds are also obtained from botanical sources. In most embodiments the botanical sources of the compounds may be one or more of Balsamo tree, Canaleta tree, Ajo de Monte, and Uña de Gato, such as tree bark, leaves, stems, roots, vines, sap, and/or inner bark. In many embodiments, the disclosed compositions may be useful in treating and/or preventing a viral infection and/or eliminating a virus from a subject. In many embodiments, the viral infection is caused by a coronavirus, for example SARS CoV-19. In many embodiments, the amount of the first, second, third, and/or fourth solvent may be between about 2 and about 150 ml, and the solvents may be combined or separate, once or more times per day, for one or more days.

Also disclosed are methods of preventing, treating, and/or curing a viral infection in a subject in need thereof, comprising administering at least a first compound to the subject on a first day. In many embodiments one or more of a second, third, and fourth compound may be administered to the subject on the same first day. In many embodiments, the subject may be administered one or more doses of each compound per day. In many embodiments, the method may include administering one or more of the first, second, third, and fourth compounds on subsequent days, for example the second day, the third day, the fourth day, the fifth day, the six day, the seventh, or more. In some embodiments, the first compound is administered on the first day and the second compound is administered on the second day, for example a compound from the Balsamo tree on the first day and a compound from the Canaleta tree on the second day. In many embodiments, the compounds may be extracted from a botanical source and administered orally as a water-based liquid medicinal composition to the subject, for example in a dosage of between about 2 ml and 150 ml, once or more times per day. In many embodiments, the botanical source for the third or fourth compound may be selected from Ajo de Monte, and Uña de Gato. In embodiments where the method may be used to cure a subject of a viral infection, the subject, after treatment, may be free of the virus by at least one method of identifying a viral infection. In embodiments wherein the subject lacks viral symptoms and/or may be at risk of exposure to a viral agent, the method may include being administered one or more of the compounds on a first day and skipping one, two, three, four, five, six, seven, eight, nine, or ten days before being administered a second dose of the same or a different compound.

Also disclosed are methods of extracting one or more medicinal compounds from a botanical source useful in the prevention, treatment, or elimination of a virus and/or viral infection. In many embodiments, the botanical source(s) of the disclosed compound(s) may be one or more of Balsamo tree, Canaleta tree, Ajo de Monte, and Uña de Gato, for example from the plant's bark, sap, stems vines, leaves, roots, and/or inner bark. In most embodiments, the compounds may be extracted with water, for example warm, boiling, or boiled water.

### BRIEF DESCRIPTION OF FIGURES

FIG. 1 shows a Balsamo tree and material harvested therefrom. Panel A shows an intact, piece of bark removed, Panel B shows a piece of bark being removed from the tree, and Panel C shows the piece cut into smaller fragments as one embodiment for obtaining one of the disclosed medicinal compounds.

FIG. 2 shows a Canaleta tree as well as a method of harvesting material therefrom to prepare one embodiment one of the disclosed medicinal compounds. Panel A shows the material being harvested from a tree, while Panel B shows a portion of the harvested material.

FIG. 3 shows leaves of the Ajo de Monte plant as one embodiment for obtaining one of the disclosed medicinal compounds. The Ajo de Monte leaves are in a pot containing water into which the medicinal compounds are extracted, and from which a medicinal vapor may be produced.

FIG. 4 is a picture of the four medicinal compositions in separate containers. The composition comprising Canaleta extract is labelled “Paleta,” the composition comprising Uña de Gato is labelled “Uña de Gato,” the composition comprising Balsamo is labelled “valtamo,” and the composition comprising Ajo de Monte is labelled “Ajo.”

### DETAILED DESCRIPTION

Disclosed herein are compositions and methods of preparing and using same in treating and preventing viral infections. The disclosed compositions comprise one or more natural or synthetic compounds/substances/molecules that may be similar or identical to those found in biological extracts, for example extracts from parts of trees and/or plants. In one embodiment, the disclosed compounds may be extracted from parts of one or more trees and or plants found in a tropical forest, for example trees and/or plants found in Ecuador.

Disclosed herein are compositions and methods for the prevention of viral infection in a subject comprising administering to the subject a composition comprising an effective dose of at least one antiviral agent. The methods and compositions described herein relate to any infective virus. In some embodiments, reference to a virus is not limited to a single virus and is inclusive of one or more infective viruses. In exemplary embodiments, a virus is at least one coronavirus, influenza, adenovirus, rhinovirus, enterovirus, poliovirus, rubella virus, paramyxovirus, rotavirus, neurotropic virus, oncovirus, picornavirus, aphthovirus, parechovirus, herpes simplex virus (HSV), human immunodeficiency virus (HIV), simian immunodeficiency virus (SIV), feline leukemia virus, picornavirus family, respiratory syncytial virus (RSV), and encephalomyocarditis virus. In various embodiments, the virus is one or more of coronavirus, Severe acute respiratory syndrome (SARS), Middle East respiratory syndrome (MERS), and Coronavirus disease 2019 (SARS-CoV-2, COVID-19 or COVID) and variants thereof.

Parts of trees and plants that may be suitable for extracting the one or more compounds include root, stem, vine, leaf, flower, fruit, bark, inner bark, trunk, sap, xylem, phloem, cambium, petal, stamen, pistil, nut, etc. In many embodiments, the disclosed compounds may be found in bark, leaves, or stems.

Tree or plant parts may be processed to create a material that may be subjected to extraction to obtain the disclosed compounds in greater concentration and/or substantially isolated from other compounds or substances. In some embodiments, processing may include scraping, cutting, shredding, grinding, etc. In some embodiments, the tree part, plant part, or processed material thereof, may be, prior to or during extraction, treated by various methods. In some embodiments, treating the material may aid in extracting the compound. In many embodiments, treating the material may include heating, steaming, boiling, etc.

The disclosed medicinal compounds may be extracted from the part or material by combining the material with a solvent, for example water. Other suitable solvents may include polar or non-polar solvents, for example acetone, chloroform, distilled water, ethyl acetate, ethanol, hexane, and methanol. In many embodiments, boiling or heating the solvent may aid in extracting the medicinal compound from the material into the solvent. In many embodiments, heated or boiling water may aid in extraction of the medicinal compounds from botanical material. The compound may be extracted as the temperature of the solvent is raised or maintained by heating for about 0 to 60 minutes. In some embodiments the compound is extracted by boiling the material for from 0-60 minutes. In some embodiments the material may be extracted as the temperature of the solvent rises to boiling, for from 0-60 minutes, and further extracted during boiling for 0-60 minutes. For example, the bio material may be added to a solvent before or during heating, and then continued to boil for 0-60 minutes, for example 5-10 minutes.

The disclosed compositions and compounds may be administered by various methods. In some embodiments, administer may refer to providing the disclosed compounds or compositions to a patient, wherein the therapeutic composition is formulated for oral, nasal, topical (including buccal and sublingual), inhalation, insufflation, transmucosal, or intravenous administration. In some embodiments, the therapeutic compositions can be formulated for different routes of administration at the same time or within the same administration

### Medicinal Composition

The disclosed compounds may be combined with various solvents and/or pharmaceutical compositions. In many embodiments, the disclosed compounds may be in a medicinal composition, comprising one or more solvents, for example water. In some embodiments, the disclosed treatment may include administering one or more medicinal compositions to a subject in need of treatment for or prevention of a viral infection. In many embodiments, the medicinal composition may be an extract from a botanical material. In some embodiments, the disclosed botanical extracts may comprise one or more botanical compounds. In many embodiments, the botanical extracts may be prepared from one or more of a tree or plant growing in a rain forest, for example in Ecuador. In various embodiments the plants are selected from Balsamo, Orenamongo, Myroxylon balsamum, Canaleta, Canalete, Awencatowe, Aspidosperma rigidum, Ajo de Monte, Mansoa alliacea, Mansoa standleyi, Wigayen, Uña de Gato, Engawene, Uncaria tomentosa, Uncaria guianensis, and combinations thereof.

### Balsamo/Orenamongo

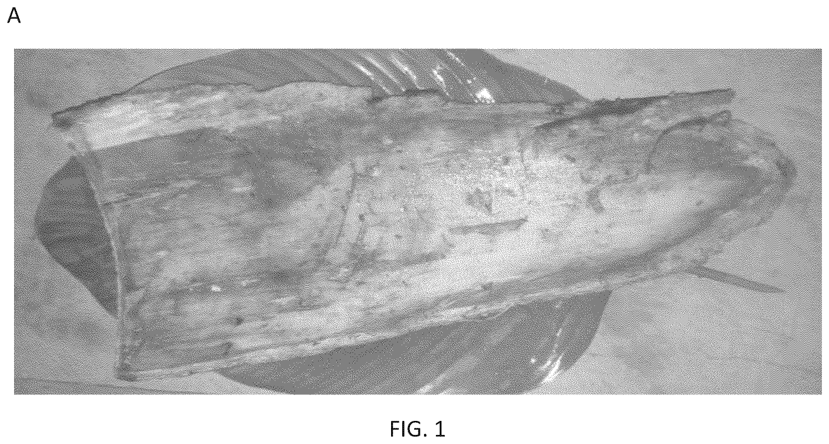

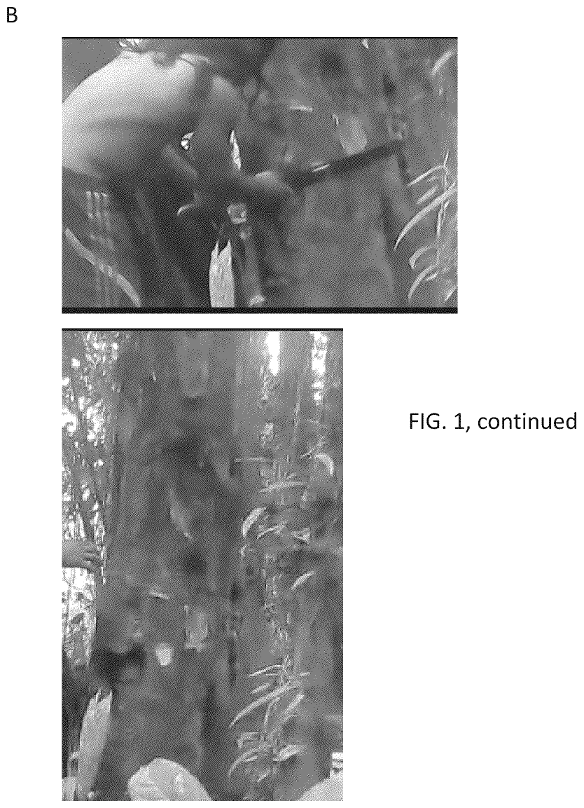

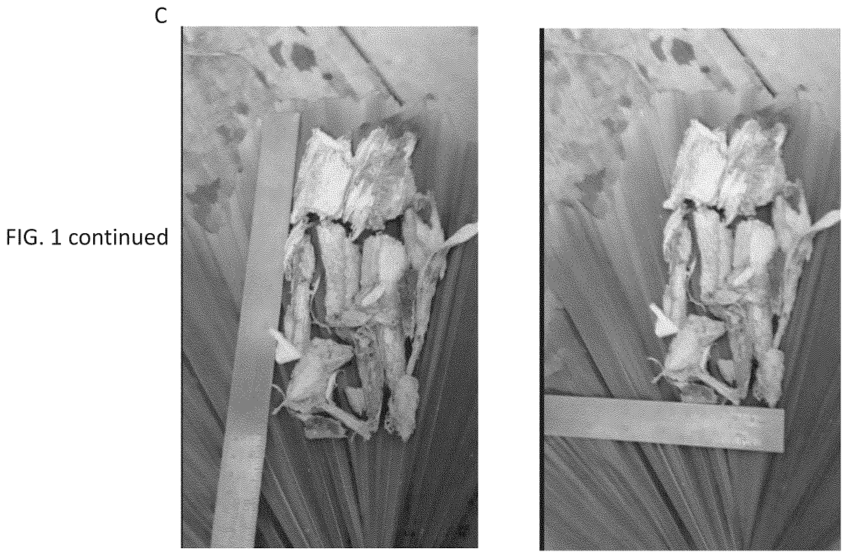

One or more of the disclosed compounds may be obtained from the tree Myroxylon balsamum, which may be referred to as Balsamo (in Spanish) and/or Orenamongo (in the Huaorani/Waorani language; note that Huaorani and Waorani are interchangeable spellings). In other embodiments, the disclosed compounds may be obtained from one or more other species of Myroxylon. In most embodiments, the compounds or substances from the Balsamo are found in and obtained from the bark. The bark may be removed from the tree in various way, for example by tool, such as a machete. In most embodiments, a large piece of bark may be removed, for example a piece measuring from about 1-5 cm thickness, 10-30 cm length, and 5-15 cm width. In many embodiments, the bark may be prepared by removing and discarding an outer layer or surface that may contain dirt and/or other contaminants, for example by scraping and/or grating that outer surface or layer, and in many cases discarding a thin, outer surface of the bark. In some embodiments, a liquid or sap may ooze from the inner surface of the bark after it has been cut or scraped. In some embodiments, the sap or liquid may be a source of the one or more compounds. A sap or liquid may also be obtained by scraping the outer surface of a root until the sap/liquid oozes from the root. The root may be found above or below the surface of the ground. FIG. 1 shows a Balsamo tree with pieces of bark being removed (Panel B), removed (Panel A), and pieces of bark (Panel C).

Balsamo bark may be processed in various ways to obtain a composition comprising the disclosed compounds and substances. In one embodiment, the bark may be boiled in a solvent, for example water, for about 10 minutes, for example more than about 0 minute to less than about 60 minutes.

Various concentrations of the Balsamo compound(s) may be obtained from the Balsamo bark. In most embodiments, approximately 100-600 cm³ of bark may be extracted in about 1.0-2.0 L of solvent (e.g. water). In one embodiment, a piece of bark approximately 22 centimeters long and 8 centimeters wide, with the outer surface scraped/grated (to remove dirt and/or contaminant), may be reduced in size by cutting into smaller pieces. The small pieces may then be added to approximately 1.5 liters of water, which may then be brought to a boil, and maintained at a boil for about 0 to 60 minutes, for example 5-10 minutes, to create a raw extracted liquid composition. The small pieces of bark may be removed from the raw extracted liquid composition to produce a liquid medicinal composition. As noted above, the botanical compounds may be extracted from botanical material or created by chemical synthesis. Synthetic forms of the disclosed medicinal compounds may be identical or similar, for example similar compounds may be synthesized with adducts or functional groups that may enhance efficacy of the compound and/or provide it with enhanced or new characteristics.

In some cases, Balsamo material may have a strong odor. In these cases, material from the Balsamo may be cooked/boiled to help diminish the odor. This diminished odor may render the extract composition more palatable and ingestible. As noted above, sap or liquid that oozes out upon removal or scraping of the bark from the trunk may also be obtained from roots, which may, in certain cases, be located above ground. In some cases, peccary have been seen extracting this sap/liquid by scraping the root, and then using their snout to apply to sap/liquid to a wound or infection. In some cases, animals such as peccary and armadillos may scrape the Balsamo root and smell/sniff the liquid/sap to treat wounds. The liquid/sap may also be used directly by humans, without cooking, as a topical treatment for skin conditions, problems, and injuries.

### Canaleta/Awencatowe

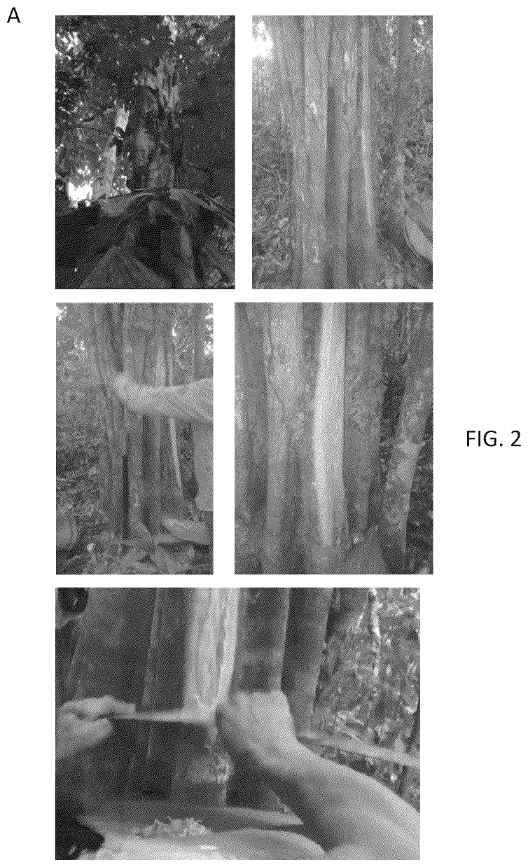

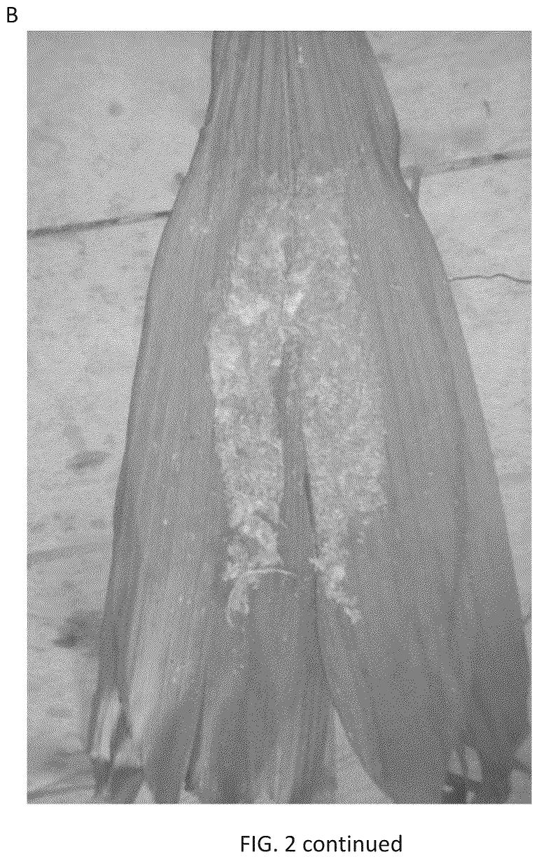

One or more of the disclosed compounds may be obtained from the tree Aspidosperma rigidum, which may be referred to as Canaleta or Canalete (in Spanish), and/or Awencatowe (Huaorani). In other embodiments, the disclosed compounds may be obtained from one or more other species of Aspidosperma. In most embodiments, the compounds or substances from the Canaleta may be found in and obtained from the bark. The bark may be removed from the tree in various way, for example by tool, such as a machete. In many embodiments, the bark may be scrapped and/or grated by machete. The grated pieces/scrapings may be transported away from the tree for further processing/preparation. FIG. 2 shows pictures of various stages of collecting material from Canaleta tree (top of Panel A). The outer part of the bark is scraped and collected (middle of Panel A), and material may be collected onto a leaf for transport. The bottom photo in Panel A shows material scraped directly onto a leaf. Panel B of FIG. 2 shows the collected material ready for processing.

The amount of grated pieces may vary from about 1-1500 cm³. In various embodiments, as shown in FIG. 2 (Panel B), an equal volume of unpacked Canaleta material is combined with a roughly equal volume of solvent. In some embodiments, about 1500 cm³ (1.5 liters) of unpacked material may be used to prepare a raw liquid composition by combining it with between about 1 and 2 liters of solvent, for example approximately 1.5 liters of water. In most embodiments, extraction may be accomplished by boiling the raw liquid composition for between about 0 and 60 minutes, for example about 10 minutes. After boiling the liquid it may be decanted, as in most embodiments the bark pieces may settle to the bottom of the boiling vessel after the raw liquid composition cools. In some embodiments, the composition may be filtered.

### Ajo de Monte/Wigayen

One or more of the disclosed compounds may be obtained from the plant Mansoa alliacea or Mansoa standleyi, that is able to grow on the ground and/or climb trees, and which is referred to as Ajo de Monte (Spanish), and/or Wigayen (Huaorani). In other embodiments, the disclosed compounds may be obtained from one or more other species of Mansoa. In most embodiments, the compounds or substances from the Ajo de Monte may be found in and obtained from the leaves, however stems and roots, and other parts, may also contain the disclosed compounds or substances. The leaves (in some embodiments the roots and stems) may be removed from the plant in various ways, for example picking or cutting. In many embodiments, the leaves, stems, and/or roots may be transported away from the plant for further processing/preparation.

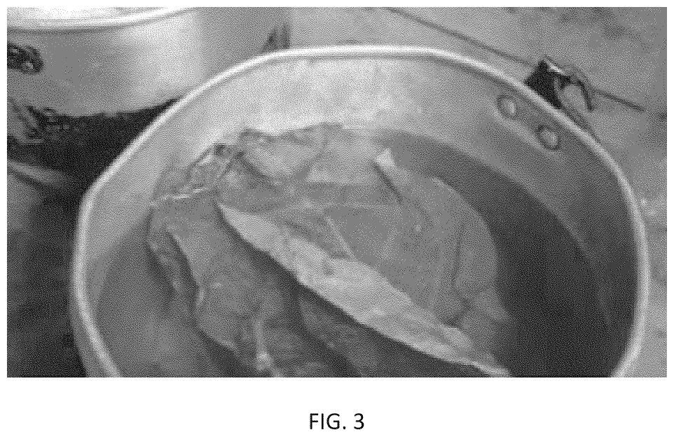

The amount of leaves may vary from about 3-20 and 0.1-5 cm³. In various embodiments, as shown in FIG. 3, about 5-10 leaves, for example about 8 leaves may be used to prepare a raw liquid composition by combining it with between about 1 and 2 liters of solvent, for example approximately 1.5 liters of water. Boiling time may preferentially be about ten minutes or from about 0 to 60 min. In most embodiments, extraction may be accomplished by boiling the raw liquid composition. After boiling the liquid, the leaves may be removed when the raw liquid composition cools.

In some embodiments, the vapor from the boiling raw liquid composition may be inhaled by the subject in need of treatment for a viral infection, but the composition after it has been prepared is, in most cases, not re-boiled for this purpose.

### Uña de Gato/Engawene

One or more of the disclosed compounds may be obtained from the plant or woody vine, Uncaria guianensis or Uncaria tomentosa, which may be referred to as Uña de Gato (Spanish) or Engawene (Huaorani). In other embodiments, the disclosed compounds may be obtained from one or more other species of Uncaria. In most embodiments, the compounds or substances from the Uña de Gato may be found in and obtained from the inner part of the stems or vines of the plant, but may also be found in other parts. The outer part of the stem may be removed from the vine by peeling the exterior. In many embodiments, the remaining, inner part of the stem, material may be cut into short pieces between about 1 cm and 15 cm in length. In many embodiments, about 1.0 and 2.0 meters of inner part, for example 1.5 meters, may be combined with about 1.0 and 2.0 liters of solvent, for example about 1.5 liters of water. The water may be boiled for from 0 min to 60 min, in many embodiments about 10 minutes. Additional water may be added to prevent thickening of the water, which will achieve a reddish-orange color. After the raw liquid composition cools the solid pieces are removed.

### Subject

A subject is any person that may be exposed to, has been exposed to, is at risk of exposure to, or is suffering from one or more symptoms associated with a viral infection. Subject and patient, may be used interchangeably to refer to a human or other mammal in need of, receiving, or having received prophylactic or therapeutic treatments as disclosed herein. A patient or subject may refer to an animal, such as a human, cow, horse, sheep, lamb, pig, chicken, turkey, quail, cat, dog, mouse, rat, rabbit or guinea pig. The animal can be a mammal such as a non-primate and a primate (e.g., monkey and human). In one embodiment, a patient is a human, such as a human infant, child, adolescent, or adult.

Viral infections include infections of DNA or RNA-based pathogenic viruses, for example, without limitation, influenza or coronavirus. In many embodiments, the virus is SARS-CoV-2, the causative agent of COVID-19. Symptoms associated with the disclosed viral infections include fever, chills, weakness, muscle ache/pain, joint ache/pain, headache, confusion, chest pain/pressure, drowsiness, shortness of breath, cough (productive and dry), bluish tint to lips or face, vomiting, diarrhea, sore throat, congestion, nasal discharge, loss of smell, weight loss, and loss of taste. In some embodiments, fever may be associated with a body temperature greater than about 98° F. or 37° C.

Subjects of various ages may be treated by the disclosed methods and composition. In many embodiments, subject may be children, young adults, middle-aged, or aged. In many embodiments, a child is anyone about 10 years or younger.

### Treatment

The terms “treat”, “treating” and “treatment” refer to eliminating, reducing, suppressing, or ameliorating, either temporarily or permanently, partially or completely, a clinical symptom, manifestation, or progression of an event, disease or condition associated with the disorders and diseases described herein. As is recognized in the pertinent field, methods and compounds employed as therapies may reduce the severity of a given disease state, but need not abolish every manifestation of the disease to be regarded as useful. Similarly, a prophylactically administered treatment need not be completely effective in preventing the onset of a condition to constitute a viable prophylactic method or agent. Simply reducing the impact of a disease (for example, as disclosed herein, reducing symptoms associated with infection such as fever, chills, pain, confusion, shortness of breath, headache, joint soreness, muscle soreness, weakness, dry cough, vomiting, weight loss, diarrhea, etc. and/or reducing the number or severity of associated symptoms, or by increasing the effectiveness of another treatment, or by producing another beneficial effect), or reducing the likelihood that the disease will occur or worsen in a subject, is sufficient. One embodiment of the invention is directed to a method for determining the efficacy of treatment comprising administering to a patient therapeutic treatment in an amount, duration, and repetition sufficient to induce a sustained improvement over pre-existing conditions, or a baseline indicator that reflects the severity of the particular disorder.

Viral infection may refer to a verified presence of a viral particle, anti-viral antibody, viral antigen, and/or viral nucleic acid in a subject using viral diagnostic tests known to those skilled in the art (e.g. immunoassays such as ELISAs, Western blot, and lateral flow assays; and PCR). In exemplary embodiments, viral infection may refer to COVID (COVID-19; CoV-19, coronavirus) infection refers to a verified presence of anti-COVID antibody, COVID particles, COVID peptides, or COVID nucleic acid. In some instances, an COVID patient may be an individual who has detectable anti-COVID antibody, COVID nucleic acids, or COVID particles in their blood, or has recently been in close contact with an individual with such compounds in their blood.

Compounds may be delivered by various methods to a subject suffering from or at risk of suffering from symptoms associated with viral infection. In many embodiments, the disclosed compounds may be delivered by ingestion of one or more liquid compositions. In various embodiments, the amount of compound and/or volume of liquid composition containing the compound may be referred to as a dose. The dose may vary depending upon the status of the subject, for example age, sex, degree of exposure, severity of symptoms, etc. In some embodiments, the dose given for prophylactic purposes and/or to subjects that are asymptomatic may be less than the dose provided to a subject with symptoms, and/or the dosing schedule may be less for prophylactic purposes.

The disclosed composition(s) may be useful in preventing infection and/or symptoms associated with infection. In many embodiments, if the subject has been exposed to the virus and/or exposure is possible (for example the subject is planning to travel, especially to an area with a dense and/or infected population), the disclosed composition(s) may be administered periodically. In some embodiments the composition(s) is administered daily, every other day, every third day, every fourth day, fifth day, sixth day, seventh day, or more, for example every 10^{th} day or longer. In many embodiments, the frequency of administration may be altered or stopped after a period of 2 to 7 days, 1 to 3 weeks, or 1 or more months. In many embodiments the frequency may be reduced or increased, depending upon subject's condition improving or worsening. In many embodiments, the disclosed compositions are administered to a patient every other day to prevent infection and/or symptoms. Dosing for subjects may be from about 2 to about 150 ml per day per composition, in one example about 15 ml or about 50 ml. In some embodiments, the dose may be about 5 ml of the one or more medicinal compositions once, twice, or more times per day. In most embodiments, the dosing for prophylactic administration is lower and less frequent than for treating a subject known to be infected. In some embodiments, the prophylactic dose may be about 5 ml to about 30 ml of a first compound on a first day, about 5 ml to about 30 ml of a second compound on a second day, and about 5 ml to about 30 ml of a third and/or fourth compound on subsequent days. In one embodiment, if a subject has been exposed, or believes exposure has occurred, 10 ml of a medicinal composition may be administered for 1 or more days. In most embodiments, prophylactic dosing (dose size and/or timing) for a child (for example a patient less than about 10 years of age) may be about ½ the adult dose.

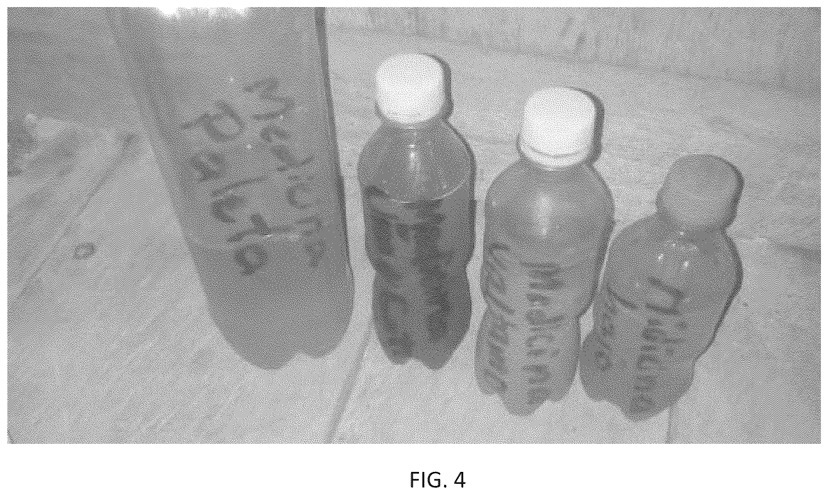

FIG. 4 shows four medicinal compositions that may be useful in treating COVID-19. In one embodiment, the subject may be treated with a compound from the Balsamo tree on the first day, and a compound from the Canaleta tree on the second day.

The disclosed compositions are useful in reducing symptoms and/or eliminating viruses in persons suffering from a viral infection or believed to be suffering from a viral infection. Eliminating a virus or an infection from a subject, as used herein, may be referred to as ‘curing’ a subject, for example wherein a subject that has tested positive for the virus (by DNA, RNA, protein, and/or antibody analysis) has no detectable virus (by the same or a different method) after treatment. In these subjects, adults may be administered about 2 ml to about 150 ml per composition per day as a single dose or separated into multiple doses. In some embodiments the subject may be administered the disclosed compounds once, twice, three, or more times a day. In some embodiments, for example, an adult may be administered about 5 ml to about 150 ml of each medicinal composition each day. In many embodiments, (for example where the dose is 15 ml/day) 5 ml is administered 3 times per day as a 5 ml dose. In other embodiments, a 5 ml/day dose may also be divided into 3 or more smaller doses for administration throughout the day—for example about 2 ml three times per day. In some embodiments, the dose may be about 10 ml to about 30 ml of a first compound on a first day, about 10 ml to about 30 ml of a second compound on a second day, and about 10 ml to about 30 ml of a third and/or fourth compound on subsequent days. In other embodiments, greater than 5 ml, for example 10 ml to about 150 ml, of a first composition comprising a first compound may be administered on a first day, and greater than 5 ml, for example 10 ml to about 150 ml, of a second composition comprising a second compound may be administered on the second day. In one embodiment, the subject may be treated with a compound from the Balsamo tree on the first day, and a compound from the Canaleta tree on the second day. The compositions may be administered from 1-5 times per day, for example 3 times/day. The treatment may last from 1 to 6 days, or more, in many embodiments the treatment is between 3 and 5 days, or more. Children suffering from or believed to be suffering from a viral infection may be administered less than the adult dose, for example in one embodiment a child may be administered from about 2 ml to about 45 ml per composition, for example about 2 ml to about 4 ml of one or more medicinal composition. The treatment may last from 1 to 6 days, or more, in many embodiments the treatment is between 3 days and 5 days, for example 3 days of treatment, 4 days of treatment, or 5 days of treatment. In many embodiments, the term “recovered,” in relation to a patient having symptoms of a viral infection, may be used to describe a significant reduction in symptoms and/or the severity of symptoms. In many embodiments, elimination of a virus may be demonstrated by one or more test results showing a lack of viral particle, protein, or DNA in a sample taken from the patient.

The term “effective amount” refers to an amount of a composition of the present disclosure or other active ingredient sufficient to provide a therapeutic or prophylactic benefit in the treatment or prevention of a disorder, disease, or condition, or to delay or minimize symptoms associated with same. Further, a therapeutically effective amount with respect to a compound of the invention means that amount of therapeutic agent alone, or in combination with other therapies, that provides a therapeutic benefit in the treatment or prevention of a disease. Used in connection with a compound or composition of the present disclosure, the term can encompass an amount that improves overall therapy, reduces or avoids symptoms or causes of disease, or enhances the therapeutic efficacy or synergies with another therapeutic agent. The phrase “therapeutically effective amount” means an amount of a compound or composition of the present invention that (i) treats the particular disease, condition, or disorder, (ii) attenuates, ameliorates, or eliminates one or more symptoms of the particular disease, condition, or disorder, or (iii) prevents or delays the onset of one or more symptoms of the particular disease, condition, or disorder described herein. In the case of viral infection, the therapeutically effective amount of the drug may reduce the number of cells infected by the virus, reduce the number of viral particles,

The term “about” or “approximately” means an acceptable error for a particular value as determined by one of ordinary skill in the art, which depends in part on how the value is measured or determined. In certain embodiments, the term “about” or “approximately” means within 1, 2, 3, or 4 standard deviations. In certain embodiments, the term “about” or “approximately” means within 30%, 25%, 20%, 15%, 10%, 9%, 8%, 7%, 6%, 5%, 4%, 3%, 2%, 1%, 0.5%, or 0.05% of a given value or range. Whenever the term “about” or “approximately” precedes the first numerical value in a series of two or more numerical values, it is understood that the term “about” or “approximately” applies to each one of the numerical values in that series.

### Examples

Subjects Treated with Disclosed Medicinal Compositions

Patient 1: Elderly male was very ill and seemed near death. With symptoms including weakness, fever, headache, dry cough, heart palpations, and difficulty breathing. His illness prompted his relatives to obtain the disclosed medicinal compositions (Patients 1- and Patient 7 were treated with medicinal compositions derived from each of Balsamo tree Myroxylon balsamum, Canaleta tree—Aspidosperma rigidum, Ajo de Monte—Mansoa alliacea, and Uña de Gato—Uncaria guianensis) to save his live. Patient 1 was treated for 4 days, and showed improvement in symptoms on Day 5. Patient 1 was deemed to have “recovered” on Day 6—after five days of treatment.

Patient 2: Elderly female was very ill and seemed near death. With symptoms including weakness, fever, muscle pain/body aches, headache, and dry cough. Patient 2 was treated for 4 days, and showed improvement in symptoms on Day 5. Patient 2 was deemed to have “recovered” on Day 6—after 5 days of treatment.

Patient 3: Male, approx. 64 years old, was very ill and believed he would die. With symptoms including weakness, fever, headache, dry cough, vomiting, weight loss, and diarrhea. Following 3 days of treatment, Patient 3 showed improvement in symptoms on Day 4. Patient 3 was deemed to have “recovered” on Day 5—after 4 days of treatment.

Patient 4: Male, approx. 24 years old was very ill and believed he would die. With symptoms including weakness, fever, vomiting, loss of sense of taste, coughing, headache, and muscle pain/body aches. Patient 4 was deemed to have “recovered” on Day 4—after 3 days of treatment.

Patient 5: Male, approx. 14 years old, had a fever and was very ill but less weak than Patients 1-4. Patient 5 was deemed to have “recovered” on Day 4—after 3 days of treatment.

Patient 6: Female, approx. 23 years old, was very ill and believed she would die. With symptoms including weakness, chills, fever, vomiting, diarrhea, dry cough, headache, weight loss and muscle aches/body aches. On Day 1 of symptoms Patient 6 took one paracetamol/acetaminophen, but symptoms persisted. On the morning of Day 2, Patient 6 took about 110 ml to about 150 ml Balsamo, which resulted in reducing fever, but she continued to suffer from diarrhea, vomiting, headache and body aches. On the morning of Day 3, Patient 6 took about 110 ml to about 150 ml Canaleta, and was significantly recovered by that afternoon, although weakness and weight loss persisted. Patient then began to eat, and on Day 8, Patient 6 was fully recovered.

Patient 7: Male, approx. age 28 years old with symptoms including headache and fever. Patient 7 was deemed to have “recovered” on Day 4—after 3 days of treatment.

Patient 8: Female, approx. 30 years old with symptoms including fever and dry cough. Patient 8 was tested for COVID-19 and was positive. On the first day of treatment, she was administered about 10 ml of compound from the Canaleta tree and about 10 ml of compound from the Balsamo tree. Patient 8 received treatment for the next four days comprising about 10 ml of the compound from the Balsamo tree in the morning. She was deemed to have “recovered” on Day 6. Thereafter, Patient 8 tested negative 5-8 days after initial positive test.

Patient 9: Female, approx. 5 years of age tested positive but asymptomatic. Patient 9 was treated with the same regimen as Patient 8, but at half doses—i.e. 5 ml instead of 10. She also tested negative with Patient 8.

1-70. (canceled)

71. A composition for treating a viral infection comprising:
a first compound in a first solvent, wherein the first compound is a botanical compound obtained from botanical bark material of a Balsamo tree; and
a second compound in a second solvent, wherein the second compound is a botanical compound obtained from a botanical leaf material of Ajo de Monte.

72. The composition of claim 71, including a third compound in a third solvent and/or a fourth compound in a fourth solvent, wherein the third and/or fourth compounds are botanical compounds obtained from botanical material selected from Canaleta tree bark and/or Uña de Gato vines.

73. The composition of claim 71, wherein the composition is useful in treating or preventing a COVID-19 viral infection and/or eliminating the COVID-19 virus in a subject.

74. The composition of claim 72, wherein the composition is useful in treating or preventing a COVID-19 viral infection and/or eliminating the COVID-19 virus in a subject.

75. The composition of claim 73, wherein the first solvent and the second solvent are water, and the botanical compounds are extracted by heating the botanical material the water.

76. The composition of claim 74, wherein the first solvent, the second solvent, and the third solvent and/or fourth solvent are water, and the botanical compounds are extracted by heating the botanical material the water.

77. The composition of claim 71, wherein the first solvent and the second solvent are combined.

78. The composition of claim 72, wherein the first solvent, the second solvent, and the third and/or fourth solvents are combined.

79. A method of preventing, treating, or curing a viral infection in a subject in need thereof, comprising:
administering a first botanical compound to the subject on the first day, wherein the first botanical compound is obtained from Ajo de Monte leaves.

80. The method of claim 79, comprising:
administering a second botanical compound to the subject, wherein the second compound is obtained from one of Canaleta tree bark, Balsamo tree bark, or Uña de Gato vines.

81. The method of claim 80 comprising:
administering one or more of a third or fourth botanical compound, wherein the third or fourth compound is obtained from Canaleta tree bark, Balsamo tree bark, or Uña de Gato vines to the subject on the third day.

83. The method of claim 79, wherein the viral infection is COVID-19 and the compound is administered as a water-based liquid medicinal composition.

83. The method of claim 80, wherein the viral infection is COVID-19 and the compounds are administered as water-based liquid medicinal compositions.

84. The method of claim 81, wherein the viral infection is COVID-19 and the compounds are administered as water-based liquid medicinal compositions.

85. A method of preventing, treating, or curing a viral infection in a subject in need thereof, comprising:
administering a first botanical compound to the subject on the first day, wherein the first botanical compound is obtained from bark of a Balsamo tree.

86. The method of claim 85, comprising:
administering a second botanical compound to the subject, wherein the second compound is obtained from one of Canaleta tree bark, Ajo de Monte leaves, or Uña de Gato vines.

87. The method of claim 85 comprising:
administering one or more of a second, a third, or a fourth botanical compound, wherein the second, third, or fourth compound is obtained from Canaleta tree bark, Ajo de Monte leaves, or Uña de Gato vines.

88. The method of claim 86, wherein the second compound is obtained from Canaleta tree bark and administered on the first day, on the second day the first compound is administered at least a second time, the viral infection is COVID-19, and the compounds are administered as water-based liquid medicinal compositions.

89. The method of claim 85, wherein the viral infection is COVID-19 and the compound is administered as a water-based liquid medicinal composition.

90. The method of claim 87, wherein the viral infection is COVID-19 and the compounds are administered as water-based liquid medicinal compositions.
